(19) United States
(12) Patent Application Publication
(10) Pub. No.: US 2018/0242996 A1
(43) Pub. Date: Aug. 30, 2018

(54) DISPOSABLE SURGICAL SWING SCALPEL WITH REUSABLE SCISSOR ACTION HANDLE

(71) Applicants: Joseph L. HUTCHISON, Lynchburg, VA (US); Calvin DUDLEY, Lynchburg, VA (US); Robert TRIMARCHE, Lynchburg, VA (US); Dilantha B. ELLEGALA, Lynchburg, VA (US)

(72) Inventors: Joseph L. HUTCHISON, Lynchburg, VA (US); Calvin DUDLEY, Lynchburg, VA (US); Robert TRIMARCHE, Lynchburg, VA (US); Dilantha B. ELLEGALA, Lynchburg, VA (US)

(21) Appl. No.: 15/558,283

(22) PCT Filed: Mar. 17, 2016
(86) PCT No.: PCT/US2016/022846

§ 371 Date: Sep. 14, 2017

(51) Int. Cl. A61B 17/3213

(52) U.S. Cl. CPC A61B 17/3213; A61B 2017/00477

(57) ABSTRACT

There is a surgical scalpel blade and a handle. The handle is constructed with a scissor action for opening an internal liner lock. The handle has a small window on one side to allow for at least actuation of the internal liner about locking mechanisms thereof. The surgical blade and attaching mechanism that may be located on the proximal end of the handle uses a blade that can quickly and safely be attached and removed from the handle via a rotating and locking mechanism. There is also a cover shield to cover a blade of the scalpel.

### CROSS-REFERENCE TO RELATED APPLICATION

This application claims priority from U.S. Provisional Application No. 62/134,049 filed Mar. 17, 2015 the disclosure of which is incorporated herein by reference.

### TECHNICAL FIELD

The present invention(s) relate to surgical scalpel blades and handles.

### BACKGROUND ART

Scalpels are regularly used by surgeons and other healthcare professionals for making incisions in a patient during an operative procedure. Typically, a certified surgical scrub technician affixes the blade to a reusable handle at the beginning of an operation, and the blade tang has to be grasped either by the user's hand or by a clamp and pulled from the handle. This procedure is not desirable because it exposes the person attempting to remove the blade to the risk of being cut if the user's hand is used to grasp the blade tang, and even if a clamp is used, this procedure is cumbersome at best. This process is similar with disassembly at the end of a surgical case.

Such awkward hand motions may lead to potential nicks or cuts that are uncomfortable and distracting. In addition, the nicks or cuts may result in blood or body fluid exposure, between the patient and the surgeon or other healthcare professionals in the operating room or elsewhere in the hospital, which may lead to the spread of infectious diseases between the patient and the healthcare professional. Concern over this situation has become especially acute because of diseases such as acquired immune deficiency syndrome, i.e. AIDS, and hepatitis.

While surgical gloves aid in reducing the chances of being cut during a surgical procedure, these gloves are not foolproof, and even when two sets of gloves are utilized, full protection is not afforded to the healthcare provider because the scalpel blade can still cut through both sets of gloves. Also, utilizing two sets of gloves reduces finger dexterity by the surgeon and thus is distracting to the surgeon and can interfere with the intended surgical procedure.

### DISCLOSURE

### Technical Problem

In view of the need for a surgical scalpel that can prevent or at least minimize the chances of accidental nicks or cuts during assembly and disassembly, numerous surgical scalpels have been designed. Unfortunately, these designs are deficient because they are cumbersome, difficult to use, and may cause unwanted shielding or exposure of the scalpel blade prior to the need for it. In addition, these prior designs are deficient because they do not provide a safe and effective way for the blade to be attached and removed from the handle before or after the surgical procedure.

### Solution to Problem

In accordance with an aspect of the present invention(s), there are improvements made to the handle. The handle is constructed with a scissor action for opening an internal liner lock, such as to allow for cleaning. The external handle has a small window on at least one side to allow for rotational movement of the internal liner locking mechanism. A surgical blade and attaching mechanism is located on the proximal end and uses a blade that can quickly and safely be attached and removed from the handle via a rotating, locking mechanism. The handle may be constructed of a reusable metal material sufficiently strong enough to withstand repeated autoclave cleaning.

The handle is made from two parts. The external handle housing is fashioned with sides that are affixed at several points by multiple locking pins. There is a larger pivot pin located at the midpoint of the handle assembly to allow the internal liner to attach to the external handle parts and rotate in at least a 90 degree fashion to allow for locking of the internal liner into operational position. The handle has a grooved recess at a proximal end of the handle on the bottom side. This allows exposure of the inner liners proximal end that contains the spring arm mechanism for locking the blade into place.

There is a small window cut out of one side of the external handle, to allow for a second spring a built into the inner liner, located at the distal end of the handle to allow for the inner liner to lock into the operational position.

There are several small notches on the inner liner to act as an inner liner stop pin positions. These are located on the distal top side of the inner liner and inferior mid-point, proximal to the pivot pin of the inner liner.

There is a retainer pin affixed to one side of the handle near the proximal blade end that holds the blade. The retainer pin acts as a lever stop when the blade is engaged into the proximal end of the handle. The blade has a unique attachment point design so that it may be affixed to a cam pin and is locked into position by the lever locking cleat.

According to exemplary embodiments, there s also a surgical scalpel having a scalpel body, a slot extending through the scalpel body, and a cover shield configured to slide along a longitudinal direction of the scalpel body by attachment with the slot. There is a forward detent hole and a rear detent hole of the scalpel body into which a first ball detent lock and a second ball detent lock of the cover shield are configured to insert there into respectively. There is a position in which the cover shield is slid distal to the scalpel body is configured to reveal a position at which a blade is attached to the scalpel body and a position in which the cover shield is slid proximal to the scalpel body is configured to cover the blade, and there is a rotation of the cover shield about the scalpel body.

### Advantageous Effects of Invention

It is therefore an object of the present invention(s) to provide a safe removable surgical scalpel blade that prevents or at least minimizes the chance that the healthcare professional will be nicked or cut when the surgical scalpel is affixed to the handle.

It is still a further object of the present invention(s) to provide a surgical scalpel that allows for the safe and effective removal of the blade from the handle after use and disposal of the blade.

It is yet a further object of the present invention(s) to provide a surgical scalpel handle with a mechanism that is easy to open and close as well as to assemble and disassemble after use for sterilization, cleaning and disposal.

Yet another object of the present invention(s) invention is a uniquely designed blade handle, with a spring arm that allows for constant tension to hold the blade in place.

Yet another object of the present invention(s) invention is a uniquely designed cover shield attachable to a scalpel body to cover a blade.

### BRIEF DESCRIPTION OF THE DRAWINGS

FIG. 1 illustrates a view of an inner liner of a handle according to exemplary embodiments.

FIG. 2 illustrates a view of an inner liner attached to a portion of a handle with a back portion of the handle removed according to exemplary embodiments.

FIG. 3 illustrates a view of a back portion of a handle to which an e liner is attached according to exemplary embodiments.

FIG. 4 illustrates a view of an inner liner rotated about a pivot pin of a handle according to exemplary embodiments.

FIG. 5 illustrates a view of an inner liner attached to a portion of a handle including a back portion of the handle according to exemplary embodiments.

FIG. 6 illustrates a view of an inner liner of a handle with a rotatable blade attached thereto according to exemplary embodiments.

FIG. 7 illustrates a view of an inner liner of a handle with a fixed blade attached thereto according to exemplary embodiments.

FIG. 8A illustrates a view of a cover shield according to exemplary embodiments.

FIG. 8B illustrates a view of a cover shield slid upon a scalpel body according to exemplary embodiments.

FIG. 9 illustrates a view of a cover shield rotated about a scalpel body according to exemplary embodiments.

FIG. 10A illustrates a top-view of a scalpel body according to exemplary embodiments.

FIG. 10B illustrates a bottom-view of a scalpel body according to exemplary embodiments.

FIG. 11 illustrates a view of a scalpel body attached to a cover shield according to exemplary embodiments.

FIG. 12A illustrates a view of a spring according to exemplary embodiments.

FIG. 12B illustrates a view of an internal slide bar according to exemplary embodiments.

FIG. 12C illustrates a view of an internal slide bar according to exemplary embodiments.

FIG. 12D illustrates a view of an internal slide bar according to exemplary embodiments.

### DESCRIPTION OF EMBODIMENTS

The advantages, features and aspects of the invention will become apparent from the following description of the embodiments with reference to the accompanying drawings, which is set forth hereinafter. Therefore, those skilled in the field of this art of the present invention can embody the technological concept and scope of the invention easily. In addition, if it is considered that detailed description on a related art may obscure the points of the present invention, the detailed description will not be provided herein. The specific embodiments of the present invention will be described in detail hereinafter with reference to the attached drawings.

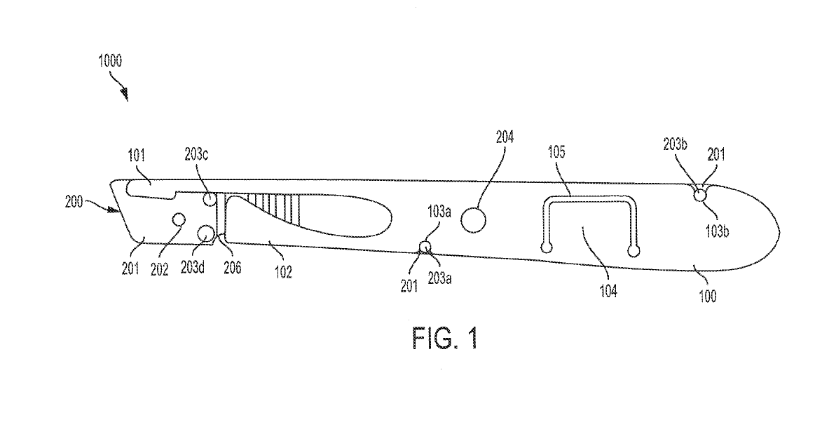

FIG. 1 illustrates a view 1000 of an inner liner 100 and an inner surface 201 of an external handle 200.

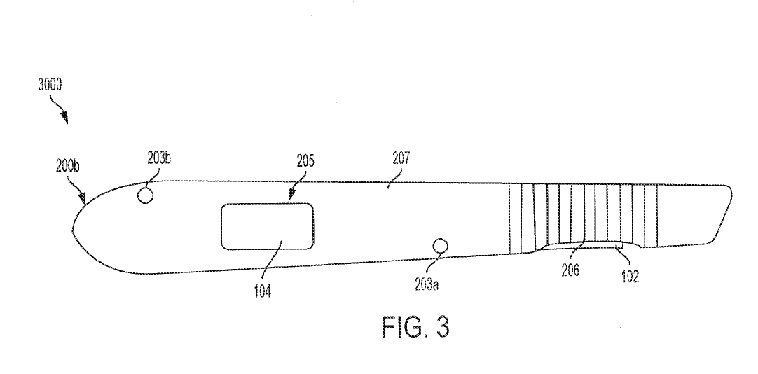

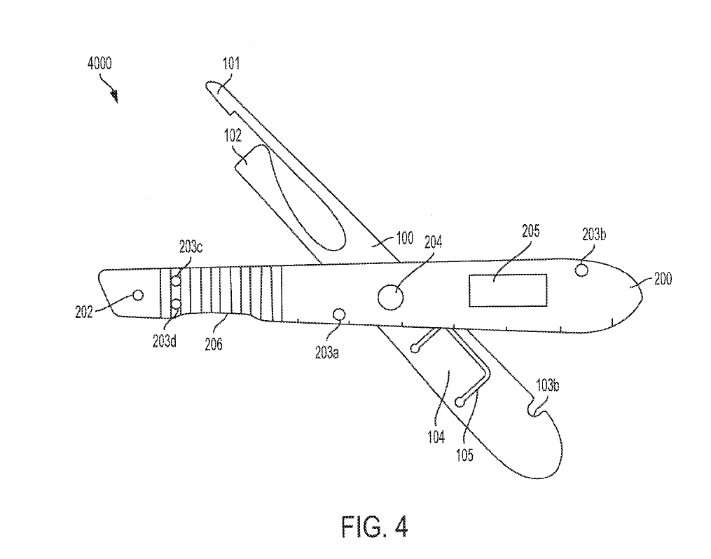

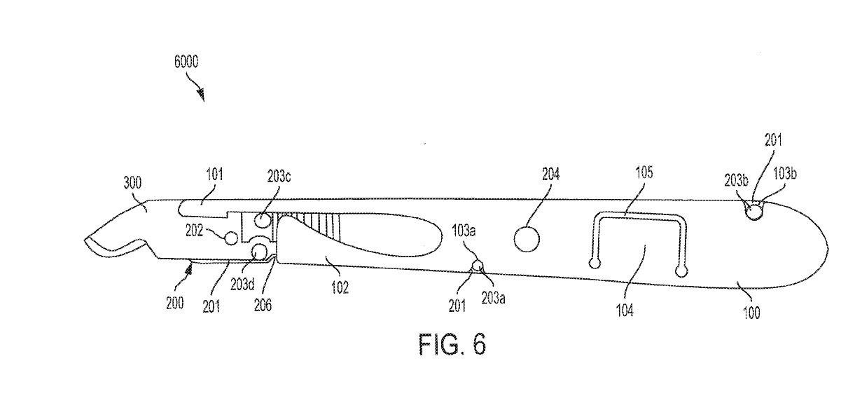

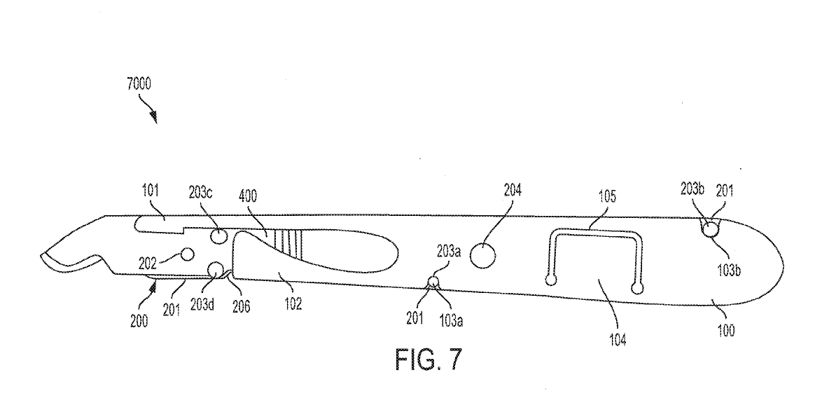

The inner liner 100 includes a locking cleat 101 and a blade retention spring arm 102 which both act to retain a blade 300 or a blade 400 as in FIGS. 6 and 7. For example, the locking cleat 101 may prevent rotation of a blade and the blade retention spring arm 102 may elastically deform and press a blade into the inner surface 201 of the external handle 200. Further, the inner liner 100 also includes an inner liner spring arm 104, in an inner liner spring arm groove 105, by which rotation of the inner liner 100 may be actuated at least through an inner liner spring arm window 205 as described with respect to FIGS. 3 and 4.

Protruding from the inner surface 201 of the external handle 200 towards the inner liner 100 are a blade pivot pin 202, a proximal locking pin 203c, a proximal locking pin 203d, an inferior midpoint locking pin 203a, a pivot pin 204 and a distal locking pin 203b.

The pivot pin 204, larger than the other pins, protrudes through the inner liner 100 such that the inner liner 100 may be attached to the external handle 200 by the pivot pin 204 and may also rotate about the pivot pin 204. Rotational motion of the inner liner 100 about the pivot pin 204 is bounded by the proximal locking pin 203c, the inferior midpoint locking pin 203a and the distal locking pin 203b. When the inner liner 100 is in line with the external handle 200, as in FIG. 1, counter-clockwise rotation of the inner liner 100, with respect to the perspective illustrated in FIG. 1, is prevented by an arm of the locking cleat 101 abutting the proximal locking pin 203c, an inferior midpoint locking pin 203a abutting an inferior midpoint locking pin groove 103a and a distal locking pin 203b abutting a distal locking pin groove 103b.

The blade pivot pint 202, proximal locking pin 203c, proximal locking pin 203d, inferior midpoint locking pin 203a, pivot pin 204 and distal locking pin 203b may be self-locking implanted cotter (SLIC) pins which may be disassembled and reassembled during maintenance of the handle and assembly of the inner liner 100 to the external handle 200 so that the inner surface 201, the locking cleat 101, and the back part 200b of the external handle 200 may be disassembled and reassembled to each other.

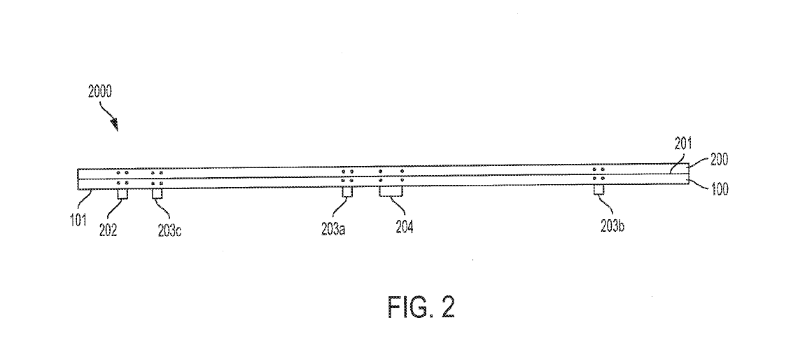

FIG. 2 illustrates a view 2000 of an external handle 200 with an attached inner liner 100. As illustrated in FIG. 2, the blade pivot pint 202, the proximal locking pin 203c, the inferior midpoint locking pin 203a, the pivot pin 204 and the distal locking pin 203b protrude through an inner surface 201 of the external handle 200 and through the inner liner 100. Further, the inner liner 100 tightly abuts the inner surface 201 of the external handle 200 according to exemplary embodiments.

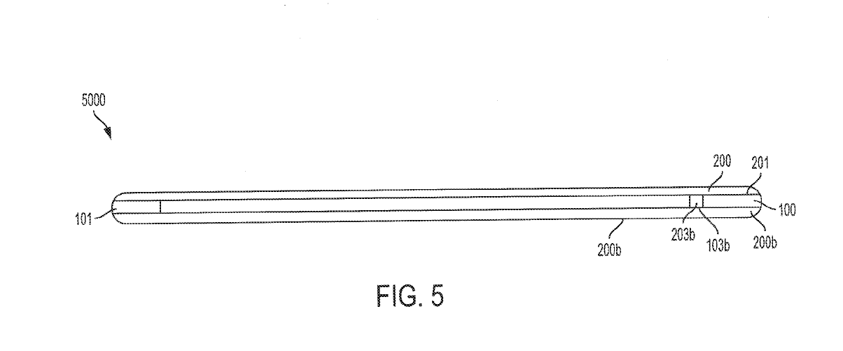

FIG. 3 illustrates a view 3000 of a back-part 200b of the external handle external handle 200. For example, FIG. 5 illustrates that the back-part 200b and the external handle 200 form a cavity there between housing the inner liner 100, FIG. 3 illustrates an outer surface 207 of the external handle 200 is illustrated. Further, there is an inner liner spring arm window 205 through which the inner liner spring arm 104 of the inner liner 100 may be actuated, such as by a user pressing against the inner liner spring arm 104 through the inner liner spring arm window 205. FIG. 3 also illustrates a grooved recess 206 of the external handle 200 and the distal locking pin 203b and inferior midpoint locking pin 203a. The grooved recess 206 allows for exposure of the blade retention spring arm 102 which may also be used to actuate the inner liner 100 by pressing against the blade retention spring arm 102 through the grooved recess 206.

FIG. 4 illustrates a view 4000 where the inner liner 100 is rotated from the external handle 200. A distal end of the inner liner 100 or a spring arm thereof is actuated by a manual inward pressure whereby the inner liner 100 will release from the proximal locking pin 203c, inferior midpoint locking pin 203a and distal locking pin 203b and rotate about the pivot pin 204, such as by a scissor-action.

As illustrated in FIG. 4, the locking cleat 101 and the blade retention spring arm 102 are rotated about the pivot pin 204 and away from the proximal locking pin 203c, proximal locking pin 203d and blade pivot pint 202 thereby opening the external handle 200 to removal or attachment of any blade thereto. Further, rotation of the inner liner 100 about the pivot pin 204 is arranged such that the distal locking pin groove 103b will contact the distal locking pin 203b and the locking cleat 101 will contact the proximal locking pin 203c when the inner liner 100 is rotated into the external handle 200, such as by a counter-clockwise rotation from the perspective of FIG. 4.

According to exemplary embodiments, clockwise rotation of the inner liner 100 in the perspective of FIG. 4 may be stopped by an abutment of the inner liner 100 with the inferior midpoint locking pin 203a.

FIG. 5 illustrates a view 5000 in which an inner liner 100 is assembled and fully within an external handle 200 and between the inner surface 201 and the back part 200b of the external handle 200. From the view 5000, the distal locking pin 203b within the distal locking pin groove 103b and the locking cleat 101 is visible. According to exemplary embodiments, the external handle 200 and the back part 200b are constructed of a reusable metal material, sufficiently strong enough to withstand repeated autoclave cleaning.

FIG. 6 illustrates a view 6000 of the inner liner 100 and external handle 200 illustrated in FIG. 1 while also including a rotatable blade 300. As illustrated in FIG. 6, the blade 300 is attached to the blade pivot pint 202 and proximal locking pin 203d and is also locked in place by the locking cleat 101 and the blade retention spring arm 102 of the inner liner 100 which latches and exerts constant tension upon a tang of the blade 300. The blade 300 may rotate about the blade pivot pint 202. The abutment of the blade 300 against the locking cleat 101 and blade retention spring arm 102 generally prevents rotation of the blade 300 which may be allowed when the inner liner 100 is rotated from the external handle 200, as in FIG. 4. The back part 200b, as in FIG. 5, may also be removed prior to removing or attaching a blade 300 to the external handle 200. The blade 300 may be removed or attached by rotating the inner liner 100 from the external handle 200, as in FIG. 4, and by sliding the blade 300 from the blade pivot pint 202 in a direction perpendicular to a plane of the inner surface 201.

FIG. 7 illustrates a view 7000 of the inner liner 100 and the external handle 200 illustrated in FIG. 1 while also including a fixed blade 400. As illustrated in FIG. 7, the blade 400 is fixed by attachment to the blade pivot pint 202, proximal locking pin 203d and proximal locking pin 203c while also abutting the locking cleat 101 and the blade retention spring arm 102 of the inner liner 100 which latches and exerts constant tension upon a tang of the blade 300. The back part 200b, as in FIG. 5, may also be removed prior to removing or attaching a blade 400 to the external handle 200. The blade 400 may be removed or attached by rotating the inner liner 100 from the external handle 200, as in FIG. 4, and by sliding the blade 400 from the blade pivot pint 202 in a direction perpendicular to a plane of the inner surface 201.

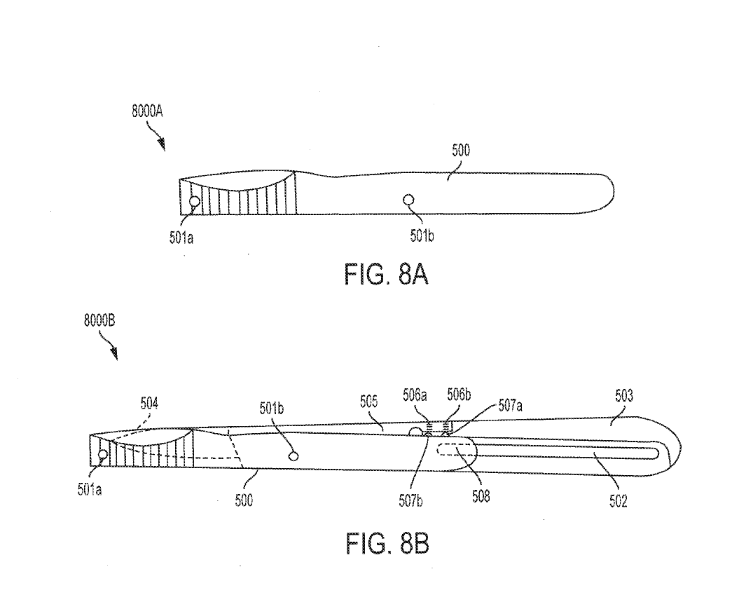

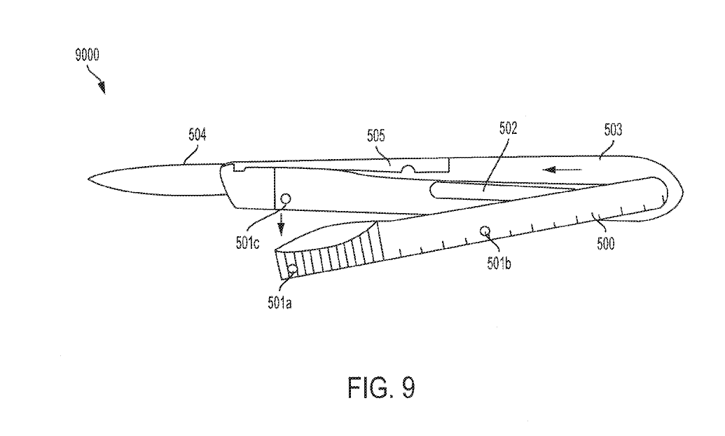

FIG. 8A states a view 8000A of a cover shield 500 including a ball detent lock 501a and a ball detent lock 501b. The cover shield 500 is configured to slide along a scalpel body 503 according to exemplary embodiments, such as in FIG. 8B, and also to rotate around the scalpel body 503, such as in FIG. 9 and to cover a blade 504. The covering of the blade 504 by the cover shield 500 may provide a safe and effective way for a scalpel to be handled.

FIG. 8B illustrates a view 8000B of a scalpel body 503 having a cover shield 500 attached thereto by an internal slide bar 508. The blade 504 is attached to the scalpel body 503 which also includes a locking spring 505 attached to the scalpel body 503 by a screw 506a and a screw 506b according to exemplary embodiments. The locking spring 505 is configured to resist rotation of the blade 504 by elastic force of the spring 507a and spring 507b.

The cover shield slot 502 is configured to allow the cover shield 500 to slide along the scalpel body 503 by attachment with the internal slide bar 508, according to exemplary embodiments. The cover shield slot 502 may extend through the scalpel body 503 along a longitudinal axis of the scalpel body 503. As described above, such as in the above-embodiments having the back part 200b of the external handle 200, the back part 200b may also have a cover shield slot 502 configured to allow for a cover shield 500 to slide thereon and to cover and uncover a blade, according to exemplary embodiments.

According to exemplary embodiments the cover shield 500 covers the blade 504 when the cover shield 500 is slid in the cover shield slot 502 proximal to the scalpel body 503 and is configured to reveal the blade 504 when the cover shield 500 is slid in the cover shield slot 502 distal to the scalpel body 503.

FIG. 9 illustrates a view 9000 illustrates a view in which a cover shield 500 is rotates about a not-illustrated internal slide bar 508 within the cover shield slot 502. FIG. 9 also illustrates a forward detent hole 501c into which the ball detent lock 501a may lock the cover shield 500 and prevent sliding therefrom according to exemplary embodiments.

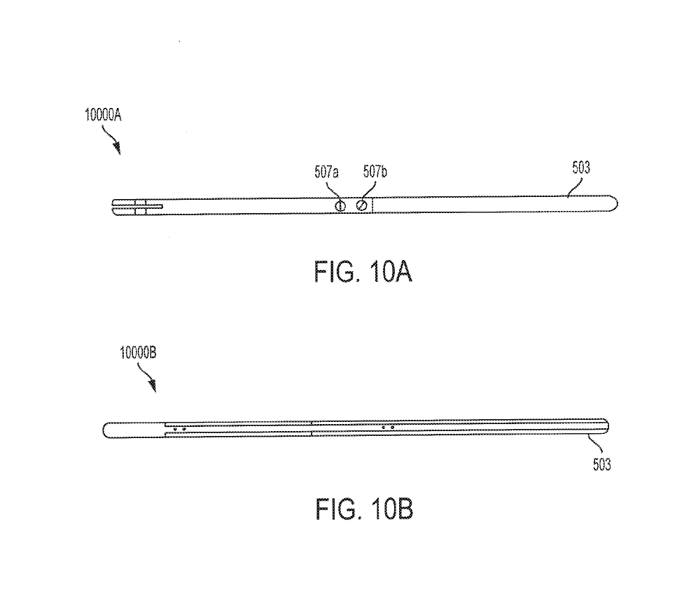

FIG. 10A illustrates a view 10000A of a scalpel body 503 from a top-down view in which the screw 506a and screw 506b may be visible according to exemplary embodiments, and FIG. 10B illustrates a view 10000B of a scalpel body 503 from a bottom-up view according to exemplary embodiments.

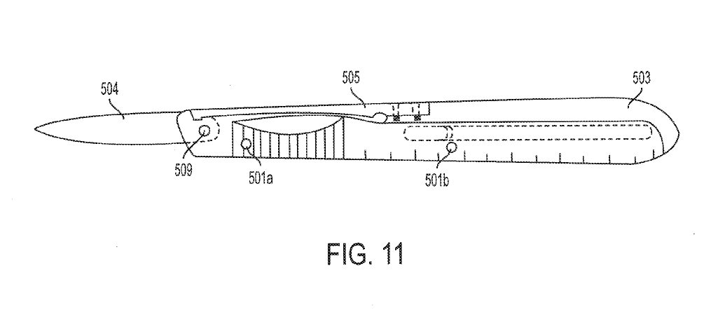

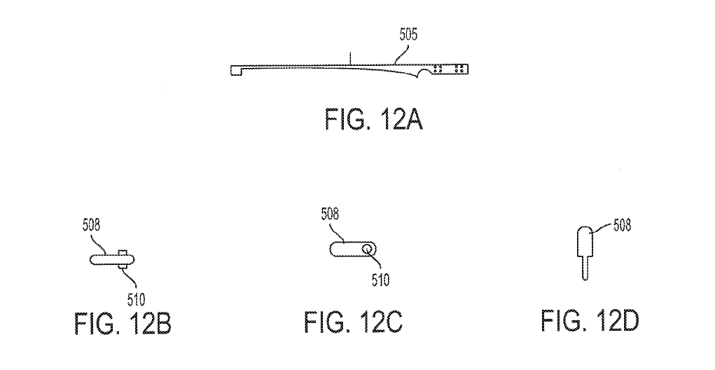

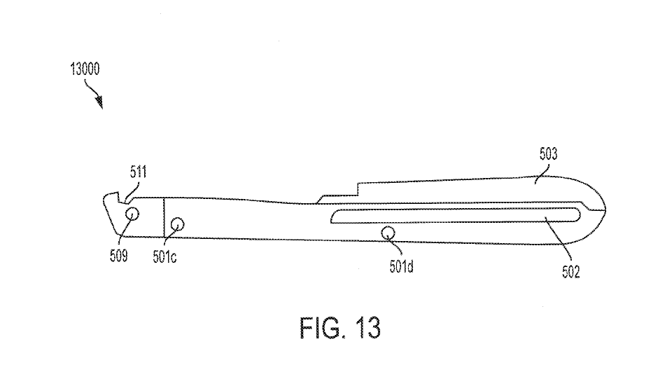

FIG. 11 illustrates a view in which the ball detent lock 501a and the ball detent lock 501b are locked into the forward detent hole 501c and the rear detent hole 501d, as illustrated in FIG. 12. FIG. 11 also illustrates a blade pin 509 about which the blade 504 may be attached.

FIG. 12A illustrates a view of a locking spring 505. FIG. 12B illustrates a side-view of the internal slide bar 508 which also includes a pin 510 according to exemplary embodiments. FIG. 12C illustrates a view of the internal slide bar 508 and the pin 510, and FIG. 12 illustrates a rear-view of the internal slide bar 508 according to exemplary embodiments.

FIG. 13 illustrates a view 13000 illustrating the scalpel body 503 which includes the forward. detent hole 501c and the rear detent hole 501d into which the ball detent lock 501a and the ball detent lock 501b may be inserted. FIG. 13 also illustrates the groove 511 of the scalpel body 503 into which the locking spring 505 may be inserted according to exemplary embodiments.

While the present invention has been described with respect to certain preferred embodiments, it will be apparent to those skilled in the art that various changes and modifications may be made without departing from the scope of the invention as defined in the following claims.

### INDUSTRIAL APPLICABILITY

Exemplary embodiments of the present application relate to a surgical scalpel having a safer detachable blade mechanism that will protect healthcare professionals from being inadvertently nicked or cut when using the device having a cover shield and/or a locking scissor action that may be disassembled to facilitate cleaning and sterilization and also prevents inadvertent partial disassembly of the surgical scalpel prior to the need to clean and sterilize the device. Although a surgical scalpel has been described herein, this is merely according to exemplary embodiments, and the features described herein may also be applied to a utility knife, a hunting knife, etc.

### REFERENCE SIGNS LIST

inner liner 100

locking cleat 101

blade retention spring arm 102

inferior midpoint locking pin groove 103a

distal locking pin groove 103b

inner liner spring arm 104

inner liner spring arm groove 105

external handle 200

inner surface 201

blade pivot pint 202

inferior midpoint locking pin 203a

distal locking pin 203b

proximal locking pin 203c

proximal locking pin 203d

pivot pin 204

inner liner spring arm window 205

grooved recess 206

blade 300

blade 400

cover shield 500

ball detent lock 501a

ball detent lock 501b

forward detent hole 501c

rear detent hole 501d

cover shield slot 502

scalpel body 503

blade 504

locking spring 505

screw 506a

screw 506b

spring 507a

spring 507b

internal slide bar 508

blade pin 509

pin 510

groove 511

### CITATION LIST

U.S. Provisional Patent Application No.: 62/006,499

What is claimed is:

1. A surgical scalpel comprising:
an external handle comprising a first surface and a second surface attached to the first surface by at least a pivot pin extending through a gap between the first surface and the second surface; and
an inner liner located within the gap, attached to both of the first surface and the second surface by the pivot pin and configured to rotate about the pivot pin.

2. The surgical scalpel according to claim 1, wherein
the inner liner comprises a blade retention spring arm configured to elastically press a blade into the first surface.

3. The surgical scalpel according to claim 1, wherein the inner liner comprises a locking cleat configured to latch into a blade and to resist rotational movement of the blade.

4. The surgical scalpel according to claim 1, wherein
the first surface comprises an inner liner spring arm window configured to expose an inner liner spring arm of the inner liner through the inner liner spring arm window.

5. The surgical scalpel according to claim 1, wherein
the first surface comprises a distal end locking pin at a distal end of the first surface,
the inner liner comprises a distal end locking groove at a distal end of the inner liner, and
the distal end locking groove of the inner liner is configured to lock into the distal end locking pin of the first surface by rotation of the inner liner about the pivot pin.

6. The surgical scalpel according to claim 1, wherein
the first surface comprises a medial locking pin at a middle of the first surface,
the inner liner comprises a medial locking groove at a middle of the inner liner, and
the medial locking groove of the inner liner is configured to lock into the medial locking pin of the first surface by rotation of the inner liner about the pivot pin.

7. The surgical scalpel, according to claim 1, wherein
a proximal end of the first surface comprises a blade pivot pin and a plurality of locking pins.

8. The surgical scalpel according to claim 7, wherein
at least one of the locking pins is configured to prevent rotational motion of a locking cleat of the inner liner.

9. The surgical scalpel according to claim 1, wherein
a distal end of the inner liner is configured to be actuated by a manual inward pressure exerted upon at least one of an inner liner spring arm and a blade retention spring arm such that the inner liner will release from a plurality of locking pins of the external handle and rotate about the pivot pin.

10. The surgical scalpel according to claim 1, wherein the first surface is configured to detach from the second surface by a release of the pivot pin from the second surface.

11. A surgical scalpel comprising:
a scalpel body;
a slot extending through the scalpel body; and
a cover shield configured to slide along a longitudinal direction of the scalpel body by attachment with the slot.

12. The surgical scalpel according to claim 11, wherein the scalpel body further comprises a forward detent hole and a rear detent hole into which a first ball detent lock and a second ball detent lock of the cover shield are configured to insert there into respectively.

13. The surgical scalpel according to claim 11, wherein in a position in which the cover shield is slid distal to the scalpel body is configured to reveal a position at which a blade is attached to the scalpel body and a position in which the cover shield is slid proximal to the scalpel body is configured to cover the blade.

14. The surgical scalpel according to claim 11, wherein the cover shield is configured to rotate about the scalpel body.
